(19) United States
(12) Patent Application Publication
(10) Pub. No.: US 2003/0017183 A1
(43) Pub. Date: Jan. 23, 2003

(54) Dermatological suspensions(micro-matrix)

(76) Inventor: David E. Pollock, Pelian Creek Crossing, FL (US)

Correspondence Address: GARDNER, CARTON & DOUGLAS, PATENT DOCKET DEPT., 321 N. CLARK STREET - SUITE 3400, CHICAGO, IL, 60610, US

(21) Appl. No.: 10/169,900

(22) PCT Filed: Jan. 8, 2001
(86) PCT No.: PCT/US01/00495

(30) Foreign Application Priority Data
Jan. 11, 2000 (US) 60175419

(51) Int. Cl. A61K 7/42; A61K 7/06; A61K 7/00

(52) U.S. Cl. 424/401; 424/59; 424/70.1; 424/74

(57) ABSTRACT

A dermatologically or cosmetically useful composition comprising a lipid phase and an anchoring agent, wherein the lipid phase and the anchoring agent are combined to form a micellular micro-matrix, and further wherein a dermatologically or cosmetically active payload may be optionally encapsulated within the micro-matrix.

### FIELD OF THE INVENTION.

The present invention relates to dermatological and cosmetic suspensions promoting efficient entrapment of a wide range of hydrophilic and/or lipophilic active or therapeutic ingredients for enhancing their delivery in topical applications.

### BACKGROUND OF INVENTION

Therapeutic or active ingredients can be included in traditional topical preparations and formulations. Traditional topical formulations, whether oil-in-water or water-in-oil or multiple emulsions, utilize emulsifiers and stabilizers to create homogenization and stabilization of the product formulation which may cause a degradation of the active payload. An active payload as used herein refers to compounds that may be incorporated into topical preparations and include therapeutic and dermatological compounds. Compounds that may be used as active payloads are further described below.

The incorporation or use of encapsulation or other types of delivery systems, such as liposomes, including oligolamellar, unilamellar and multilamellar structures. nanospheres, and others, can help protect and diminish the degradation of the active payload from emulsifiers and aggressive materials incorporated into the formulation. The use of these encapsulation or delivery systems can also improve the compatibility of active payloads with formulations, increase permeation of the active payload, minimize reaction or irritation to the skin, and allow a higher level of an active material to be added to a formulation. Additionally, the formulation shelf life may be extended. Such encapsulation and delivery systems may allow the addition of the active payload to occur at lower temperature levels than otherwise obtainable. This aspect is particularly valuable where certain active payloads may be sensitive to temperature.

The skin is comprised of the epidermis, the outer most layer, and dermis layers, that together provide a flexible support structure. The skin additionally contains the blood vessels, nerves, sweat glands, hair follicles, and sebaceous glands. The epidermis is comprised of five distinct cell layers starting with the stratum corneum or horny cell layer through to the basal cell layer.

Although the skin is very thin, the stratum corneum layer provides resistance and protection to undesired penetration of environmental insults, chemical irritants and other foreign substances. The horny cell layer is comprised of two phases: the corneocytes and the lipid matrix between the corneocytes. Over the past few decades, it has been discovered that more specifically, the lipids located between the corneocytes within the stratum corneum are responsible for the barrier function of the skin.

By looking at this layer as a brick and mortar type system, this lipid matrix between the corneocytes would act as the mortar in a brick wall. Even more recently, a closer look at this mortar, or lipid matrix, has revealed that it is comprised of parallel lamellar lipid structures. Further analysis found that these membranes consist mainly of phospholipids, ceramides and cholesterol, while the surface skin lipids are comprised of triglycerols and squalene.

The membrane structure of the lipid matrix is of main importance to the barrier function. The nature of this structure provides greater protection for the barrier function than if the same lipids were not lamellar in nature.

A dermatological suspension or formulation must be capable of protecting and delivering an active payload to a targeted area, and, under certain circumstances, may be required to penetrate the barrier layer. Several types of delivery systems have been invented throughout the years.

Liposomes provide one option for delivering select active payloads. Liposomes are microscopic spherical vesicles. Several methods are known for preparing active entrapping liposomes. One method includes depositing vesicle-forming lipids as a thin film, then slowly rehydrating with the addition of an aqueous buffer. The material to be entrapped may be included in the lipid material, if lipophilic, or in the aqueous buffer, in the case of a hydrophilic material. The end result is multilamellar vesicles (“MLVs”), where several vesicles form one inside the other in diminishing size, ranging from 0.05 to 10 microns. Liposomes with a size range of about 0.2 to 0.4 microns are generally preferred, and generally require additional processing, typically by means of homogenization, sonication or membrane extrusion. The advantages of liposomal delivery are based on the entrapment or encapsulation of the active material. MLVs are known to provide relatively poor entrapment or encapsulation hydrophilic materials. Additionally, MLVs tend to be unstable and can leak active material from the encapsulated liposome.

Another type of liposomes are unilamellar vesicles, a single phospholipid bilayer sphere encapsulating a hydrophilic material. One method of preparing unilamellar liposomes is through ultra high-shear processing. These liposomes are commonly 200 nanometers or less in size and are uniform in size. Multilamellar liposomes are essentially liposomes with a number of concentric lipid bilayers separated by a hydrophilic phase. Unilamellar liposomes have a higher efficiency level of encapsulation, when compared to multilamellar liposomes, but still may be unstable and leak the active payload. Fusion of the vesicles can also occur.

Nanospheres are very small matricial particles, typically 100 nanometers in size, and include a porous polymer, such as polystyrene or polysiloxane, that enables the nanosphere to absorb the active material and act as a micro-reservoir. Nanospheres are an aqueous suspension of, typically, acrylic copolymer nanoparticles, positively charged via specific quarternary ammonium type functionality. Nanospheres also tend to have low encapsulation efficiency.

### SUMMARY OF THE INVENTION

The present invention provides improvements over the existing delivery or entrapment systems in dermatological preparations. With appropriate agitation, achieved through standard types of production mixers such as double planetary agitation, a structure is achieved that entraps an active payload in micro-vesicles that are dispersed between parallel, lamellar micelles. Micelles are spherical structures which form when lipophilic and non-lipid materials are present in concentrations sufficient to induce the formation of such structures by simple ionic shielding effects and mass action principles. According to the principles of the present invention, such a structure may be achieved without the need for rehydration and/or high sheer agitation. The term “micro-matrix” is used hereinafter to describe the micellular structure of the present invention. The micro-matrix may be loaded with a dermatologically or cosmetically useful active payload. The micro-matrix provides several advantages in the topical delivery of therapeutic ingredients, including providing a more effective and efficient means of stabilizing readily oxidizable lipophilic and/or hydrophilic materials, improved entrapment and delivery efficiencies allowing for a higher payload level, minimization of any type of negative greasy after feel even with higher levels of lipohilic material, an ability to help restore the natural lipid layer to damaged skin and an ability to reduce transepidermal water loss.

### DETAILED DESCRIPTION OF THE INVENTION

The present invention provides a multi-component system that includes a parallel, lamellar micro-matrix micelle structure, which may optionally contain active payload material entrapped therein. Utilizing such structures, the active payload may be present in higher concentrations than its water and/or lipid solubility would otherwise dictate. The micro-matrix lamellar structure is formulated with ingredients similar to the skin lipids in order to mimic the skins natural lamellar matrix, to better combine with the skin lipids for optimum delivery of actives and so not to irritate the skin. Once the micro-matrix is delivered through the epidermis, the micelle ruptures, releasing the active payload.

The present invention includes a micro-matrix micelle comprised of a lipid phase and an anchoring agent, and a dermatological or cosmetic preparation comprised of a micro-matrix and a payload encapsulated within the lipid phase. The present invention allows for unique formulating properties, including up to a 40% lipophilic phase without an oily after-feel. This structure lowers the interfacial surface tension, while suspending oil droplets. Different than typical liposomal delivery vesicles, the composition of the present invention is alcohol free. The micro-matrix provides improved stability to the dermatological suspension and topical formulation, improved permeation of the active payload, and efficacy of the overall product with lower active payload usage levels as compared to conventional liposomal delivery vesicles.

The micro-matrix may contain active payload in an amount equal to about 1% to about 50% by weight of the micro-matrix plus payload composition, more preferably about 10% to about 25%. Active payloads may be any dermatologically or cosmetically useful substance or combination of substances, either hydrophillic or lipophillic or a combination of both. Examples of payloads that are suitable for use in the invention include but are not limited to anti-oxidants, anti-free-radicals, hydroxy acids, hydrating agents, humectants, melanin regulators, lipo-regulators, anti-acne agents, anti-aging agents, anti-wrinkle agents, sunscreen agents including ultra-violet (UV) protectors, emollients, anti-inflammatories, cooling agents, anti-bacterial agents, insect repellents, anti-dandruff agents, anti-hair loss agents, hair growth promoters, colorants including tanning agents, botanical extracts, vitamins, minerals, nutrients, and any other dermatologically active ingredient that is stable within the micellar structure through delivery

For instance, botanical extracts that may be used for the payload in the compositions of the invention include but are not limited to extracts of chamomile, lemongrass, green tea, licorice root, meadowsweet, honey, Paraguay tea, royal jelly, ginkgo biloba, aglae, red marine algae, aloe vera, coneflower, ginseng and any other dermatologically or cosmetically useful biological extract Such extracts are readily available commercially, and methods of their preparation are well known.

Examples of vitamins include vitamins and vitamin derivates, including but not limited to tocopheryl acetate, retinyl palmitate, 1-corbic acid, ascorbyl palmitate, ascorbyl glucosamine, riboflavin (vitamin b2), nicotinamide (vitamin b3), pyridoxine hcl (vitamin b6), vitamin e and any other vitamin or vitamin extract that is chemically stable within the micellar structure through delivery through the epidermis. Such vitamins and derivatives thereof are readily commercially available from a wide variety of sources.

Examples of hydroxy acids are alpha and/or beta hydroxy acids and/or poly hydroxy acids including, but not limited to, glycolic acid, lactic acid, malic acid, tartaric acid, grape ferment extract, salicylic acid, and any other hydroxy acid that is chemically stable within the micellar structure through the point of delivery. Such vitamins and derivatives thereof are readiiy commercially available from a wide variety of sources.

Suitable sunscreen agents include but are not limited to Parsol 1789, titanium dioxide, coated titanium dioxide, zinc oxide, coated zinc oxdie, ethylhexyl methoxycinnamte, benzophonene 3 or 4 or 8, PABA, and any other sunscreen agent, now known or hereafter developed, that is stable within the micellar structure through delivery.

Suitable tanning agents include but are not limited to dihydroxyacetone, lawsone and dihydroxyacetone, caramel, erythrulose, unipertan, 1-tyrosine, riboflavin, walnut extract, walnut oil, and any other tanning agent that will be stable as used in the invention.

Examples of nutritionals include, for example only and not as a limitation, wheat and/or oat protein, amino acids, hydrolyzed wheat and/or oat protein, beta glucan, gliadin, sodium hyaluronorate, sodium PCA, various humectants, shea butter, blue cypress oil, lubrajel oil, lycopene, beta carotene, uniquinone (coenzyme Q10), dimethicone, dimethiconol, pentylene glycol, certain oils including but not limited to, unsapponified avocado oil, avocado oil, pistachio oil, jojoba oil, kukui nut oil, squalane (olive and/or shark derived), and any other dermaologically or cosmetically acceptable nutritional that is stable within the micro-matrix of the invention until the point of rupture of the micelle upon delivery.

In one preferred embodiment of the invention, the micro-matrix employs an anchoring agent, for example, glycerin, or glycerin derivatives, or any composition suitable for use as an anchoring agent, which is then incorporated between the parallel lipid bilayers of the lipid phase. Glycerin, for example, acts as a hydrator of the stratum corneum by creating a reservoir of glycerin in the stratum corneum. Studies have shown high glycerin levels in formulations delivers glycerin in to the skin and increases the thickness of the stratum corneum (Appa, Y., et al., Clinical Evaluations of Hand and Body Moisturizers that Heal Skin Dryness, poster presentation at the 1992 American Academy of Dermatology. San Francisco. Dec. 6-7, 1992).

Due to the nature of the micro-matrix, a lower percentage of anchoring agent is required to effect penetration of the stratum corneum as compared to prior art compositions. The preferred micro-matrix contains about 20% to about 80% (w/w), more preferably about 40% to about 60% anchoring agent, such as but not limited to glycerin or a glycerin derivative. In addition to glycerin, other suitable anchoring agents will be obvious to those skilled in the art, and such anchoring agents are considered within the scope of the invention.

According to the principles of the present invention, the active payload is combined with the lipid phase and processed in accordance with the method of the invention, described in the examples below. The lipid phase comprises about 20% to about 90% (w/w) of the sum of the micro-matrix plus the active payload, more preferably about 25% to about 60%, and even more preferably about 30% to about 40%.

The lipid phase encapsulates the payload ingredients and is optimized to mimic the natural lipid membranes found in stratum corneum. The lipid phase preferably consists of isolated phospholipids that may be derived from soy-lecithin, triglycerides, palm oil, squaline, caprylic/capric triglyceride, and phytosterols, although other lipids may be suitable for use as the lipid phase of the invention. The lipid phase may also include compounds whose chemical structure and properties are similar to that of cholesterol, shea butter, or squalane. The lipid phase may include compounds isolated from olive oil, ceramides, ceramide III, and pentylene glycol. A preferred example of a lipid useful in the present invention is DMS concentrate, available from Kuhs, GmbH, Lagenfeld, Germany.

Compositions utilizing the invention may contain a wide variety of additional ingredients generally used in skin care preparations, cosmetics, topical pharmaceuticals, and the like.

The compositions of the present invention also provide an improved method of manufacture as compared to the manufacturing process for conventional liposomal delivery vesicles. Conventional formulations require high energy mixing,such as homogenization, and may also require a heating step. Almost any lipophilic or hydrophilic active or therapeutic ingredient or ingredients may be added directly to the micro-matrix with conventional medium shear mixing technology. According to principles of the present invention, the micro-matrix, either loaded with an active payload or unloaded, can be incorporated directly into dermatological suspensions, topical formulations and emulsions at about 5% to about 50% (w/w), more preferably about 5% to about 25%, of the overall final product. The present invention does not require any additional heating, allowing for low temperature incorporation of active payloads, although heating is an obvious variation of the invention and considered within the scope.

The following examples serve to illustrate embodiments of the present invention.

Example 1: Generic micro-matrix

| | |
|---|---|
| 1. Lipid Phase | 60.0% |
| 2. Anchoring Agent | 40.0% |

In the lab, a standard Ika type lab mixer will be sufficient. Blend 1 for 10-15 minutes to prepare lipid structure. While continuing agitation, slowly disperse in 2. Blend until visually uniform.

Example 2: Base micro-matrix

| | |
|---|---|
| 1. DMS Concentrate | 60.0% |
| 2. Glycerin, 99%, USP | 40.0% |

In the lab, a standard Ika type lab mixer will be sufficient. Blend 1 for approx. 10-15 minutes to prepare lipid structure. While continuing agitation, slowly disperse in 2. Blend for approx. 30 minutes or until visually uniform.

Example 3: Generic Micro-matrix/Payload Complex

| | |
|---|---|
| PHASE A | |
| 1. Lipid Phase | 20% to 90% |
| PHASE B | |
| 2. Anchoring Agent | 20% to 80% |
| 3. Payload composition | 5% to 50% |

In the lab, a standard Ika type lab mixer will be sufficient. Blend the lipid phase for approx. 10-15 minutes to prepare lipid structure. Phase B: Separately blend 2 & 3 for 5-10 minutes. While continuing agitation in Phase A, slowly disperse in Phase B mix. Blend for 30 minutes or until visually uniform.

Depending on the chemical nature of the payload, the final product may be either a dispersion or a solution.

Example 4: Micro-matrix with Retinol A Active Payload

| | |
|---|---|
| PHASE A | |
| 1. DMS Concentrate (lipid phase, pre-mixed by Kuhs) | 57.0% by |
| weight | |
| PHASE B | |
| 2. Glycerin, 99%, USP | 38.0% by |
| weight | |
| 3. Retinol-A (Available from BASF Corp.) | 5.0% by |
| weight | |

In the lab, a standard Ika type lab mixer will be sufficient. Blend 1 for 10-15 minutes to prepare lipid structure. Phase B: Separately blend 2 & 3 for approx. 5-10 minutes, or until visually uniform. While continuing agitation in Phase A, slowly disperse in Phase B mix. Blend for 30 minutes or until visually uniform.

Example 5: Day Cream with Retinol A Micro Matrix

| | |
|---|---|
| PHASE A | |
| 1. Deionized water | 51.9% by weight |
| 2. Polyglycerolmethacrylate (Lubrajel oil) | 1.0% by weight |
| 3. Sea algae (Seamollient) | 10.0% by weight |
| PHASE B | |
| 4. Sepigel 305 | 6.0% by weight |
| 5. Borage oil | 0.5% by weight |
| 6. Jojoba oil | 1.5% by weight |
| 7. Sesame oil | 4.5% by weight |
| 8. Sweet almond oil | 3.5% by weight |
| 9. Cetearyl octanoate | 10.0% by weight |
| PHASE C | |
| 10. Fragrance | 0.1% by weight |
| 11. Germaben II | 1.0% by weight |
| PHASE D | |
| 12. Micro-matrix with Retinol A (From Example 2) | 10.0% by weight |

Phase A: Separately, blend 1-3 until uniform. Phase B: in separate vessel, add in 4. Mix in 5 -9, one at a time. Mix well. With strong agitation, disperse phase A in to phase B and mix until uniform. Mix in Phase C. Mix in Phase D. Mix until uniform.

Accordingly, the present invention provides an improved dermatological or cosmetic suspension that utilizes micro-matrix micelles to provide improved stabilization of active payloads, permeation of the active payload into the skin and shelf-stability, the ability to incorporate greater percentages of active payloads, and minimization of skin irritation, as compared to prior art dermatological and cosmetic suspensions. In addition, the compounds of the present invention permit an improved method of manufacture, where the compositions of the invention may be prepared without homogenization or extensive shearing, and may optionally be prepared without heating.

Thus, it is apparent that there has been provided, in accordance with the present invention, a novel improved dermatological composition and method of manufacture that fully satisfies the objects, aims and advantages set forth above. While the invention has been described in conjunction with specific embodiments thereof, it is evident that many alternatives, modifications and variations will be apparent to those skilled in the art in light of the foregoing description. Accordingly, it is intended to include all such alternatives, modifications and variations as set forth within the spirit and scope of the appended claims.

What is claimed is:

1. A dermatological of cosmetic composition comprising a micellular micro-matrix, wherein the micro-matrix comprises a lipid phase and an anchoring agent.

2. The composition of claim 1, wherein the micro-matrix further comprises an active payload, wherein the active payload is removably situated within the micro-matrix.

3. The composition of claim 2, wherein the composition is added to a topical composition.

4. The composition of claim 2, wherein the micro-matrix comprises from about 1% to about 50% weight of the sum weight of the payload plus the micro-matrix.

5. The active payload of claim 2, wherein the active payload contains one or more cosmetically or dermatologically effective compounds.

6. The active payload of claim 5, wherein the dermatologically or cosmetically effective compound comprises one or more of the following:
anti-oxidants, anti-free-radicals, hydroxy acids, hydrating agents, humectants, melanin regulators, lipo-regulators, anti-acne agents, anti-aging agents, anti-wrinkle agents, sunscreen agents including ultra-violet (UV) protectors, emollients, anti-inflammatories, cooling agents, anti-bacterial agents, insect repellents, anti-dandruff agents, anti-hair loss agents, hair growth promoters, colorants including tanning agents, botanical extracts; vitamins, minerals, nutrients, and any other dermatologically active ingredient that is stable within the micro-matrix up to the point of rupture of the micro-matrix.

7. The composition of claim 1, wherein the anchoring agent comprises glycerin and glycerin derivatives.

8. The composition of claim 7 wherein the anchoring agent comprises from about 20% to about 90% weight of the micro-matrix.

9. The composition of claim 1, wherein the lipid phase comprises one or more phospholipids that may be derived from soy-lecithin, triglycerides, palm oil, caprylic/capric triglyceride, phytosterols, compounds whose chemical structure and properties are similar to that of cholesterol, shea butter, or squalane, compounds isolated from olive oil, ceramides, ceramide III, or pentylene glycol.

10. The composition of claim 9, wherein the lipid phase is DMS concentrate.

11. The composition of claim 9, wherein the lipid phase comprises from about 20% to about 90% of the micro-matrix of the invention.

12. The composition of claim 2, further comprising cosmetically acceptable compounds.

13. The composition of claim 2, further comprising dermatologically acceptable compounds.

14. A method of manufacturing a pharmaceutical or cosmetic composition comprising the steps of:
a. blending a lipid phase using a standard lab mixer until micelles form;
b. blending an effective amount of an anchoring agent and an active payload until uniform;
c. blending the lipid phase and the anchoring agent/payload mixture until visually uniform.

15. The method of claim 14 wherein heat is applied to the composition during blending.

16. A method of manufacturing a micro-matrix comprising the steps of:
a. blending a lipid phase at room temperature until micelles form, using a standard lab mixer; and
b. slowly adding an effective amount of an anchoring agent and blending until uniform using a standard lab mixer.
